(19) United States
(12) Patent Application Publication
(10) Pub. No.: US 2025/0235494 A1
(43) Pub. Date: Jul. 24, 2025

(54) THERAPEUTIC AGENT FOR OSTEOPOROSIS AND PLANTAR FASCIITIS AND METHOD OF PREPARING THE SAME

(71) Applicant: Dong Won MOON, Gyeonggi-do (KR)

(72) Inventor: Dong Won MOON, Gyeonggi-do (KR)

(21) Appl. No.: 19/023,861

(22) Filed: Jan. 16, 2025

(30) Foreign Application Priority Data
Jan. 19, 2024 (KR) 10-2024-0008775

(51) Int. Cl. A61K 36/07; A61K 35/618; A61K 35/62; A61K 35/64; A61K 35/646; A61K 36/15; A61K 36/48; A61K 36/73; A61P 19/10; A61P 29/02

(52) U.S. Cl. CPC A61K 36/07; A61K 35/618; A61K 35/62; A61K 35/64; A61K 35/646; A61K 36/15; A61K 36/48; A61K 36/73; A61P 19/10; A61P 29/02; A61K 2236/33; A61K 2236/53

(57) ABSTRACT

Provided are a therapeutic agent for osteoporosis and plantar fasciitis containing pine roots, Fomitopsis pinicola, Fomitella fraxinea, Trametes versicolor, Sorbus commixta fruits, Phellinus linteus, Lespedeza bicolor petals, Lespedeza bicolor leaves, and distilled liquor, and a method of preparing the same. The therapeutic agent has the effects of reducing the rate of decrease in bone density in patients with osteoporosis or increasing the bone density in patients with osteoporosis, significantly ameliorating heel pain in patients with plantar fasciitis, improving cognitive ability in elderly people with dementia, and alleviating various menopausal symptoms in women.

### BACKGROUND

The present invention relates to a therapeutic agent for osteoporosis and plantar fasciitis and a method of preparing the same, and more specifically, to a therapeutic agent for osteoporosis and plantar fasciitis that has the effects of reducing the rate of decrease in bone density in patients with osteoporosis or increasing the bone density in patients with osteoporosis, significantly ameliorating heel pain in patients with plantar fasciitis, improving cognitive ability in elderly people with dementia, and alleviating various menopausal symptoms in women, and a method of preparing the same.

Osteoporosis is a disease with porous bones because the bone density is lowered compared to normal bones due to rapid loss of calcium, which is the main component of the bone. Osteoporosis is a disease in which bones are greatly lost and weakened due to various causes such as menopause, aging, and use of drugs harmful to the bones and the bones are thus easily fractured even by minor impacts.

Meanwhile, the plantar fascia is composed of a strong and thick fibrous band that starts from the heel bone (calcaneus) of the sole of the foot and extends in five branches toward the front of the sole and attaches to the base of the toes. The plantar fascia helps maintain the arch of the foot and lifts the foot in a weight-bearing state, and plays an important role in the dynamics of the foot when walking. Plantar fasciitis is an overuse syndrome characterized by local inflammation that occurs during the process of supporting the arch, or a disease caused by degenerative changes in the calcaneus, which is the anatomical attachment point of the plantar fascia.

Meanwhile, various therapeutic agents are being developed to treat or alleviate osteoporosis and plantar fasciitis, and in particular, the following Patent Document 1 discloses a therapeutic agent for osteoporosis containing an astragalus extract as an active ingredient.
Patent Document 1: Korean Patent No. 10-0284657 (2001. 04. 02)

### SUMMARY OF THE INVENTION

Therefore, the present invention has been made in view of the above problems, and it is an object of the present invention to provide a therapeutic agent for osteoporosis and plantar fasciitis that has the effects of reducing the rate of decrease in bone density in patients with osteoporosis or increasing the bone density in patients with osteoporosis, significantly ameliorating heel pain in patients with plantar fasciitis, improving cognitive ability in elderly people with dementia, and alleviating various menopausal symptoms in women, and a method of preparing the same.

In accordance with an aspect of the present invention, the above and other objects can be accomplished by the provision of a therapeutic agent for osteoporosis and plantar fasciitis containing pine roots, Fomitopsis pinicola, Fomitella fraxinea, Trametes versicolor, Sorbus commixta fruits, Phellinus linteus, Lespedeza bicolor petals, Lespedeza bicolor leaves, and distilled liquor.

The therapeutic agent may further contain Cryptotympana atrata, Oxya sinuosa, Apis cerana Fabricius, Nephila clavate, Gryllus bimaculatus, Camponotus japonicus, Lumbricus terrestris, Pieris rapae, Sieboldius albardae Selys, and Acusta despecta sieboldiana.

In accordance with a further aspect of the present invention, there is provided a method of preparing a therapeutic agent for osteoporosis and plantar fasciitis including preparing mixing pine roots, Fomitopsis pinicola, Fomitella fraxinea, Trametes versicolor, Sorbus commixta fruits, Phellinus linteus, Lespedeza bicolor petals, and Lespedeza bicolor leaves with distilled liquor to prepare a first mixture (S100), filtering the first mixture to remove solid ingredients (S200), adding Cryptotympana atrata, Oxya sinuosa, Apis cerana Fabricius, Nephila clavata, Gryllus bimaculatus, Camponotus japonicus, Lumbricus terrestris, Pieris rapae, Sieboldius albardae Selys and Acusta despecta sieboldiana to the filtered first mixture to prepare a second mixture (S300), and filtering the second mixture to remove solid ingredients (S400).

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram illustrating a method of preparing a therapeutic agent for osteoporosis and plantar fasciitis according to one embodiment of the present invention.

### DETAILED DESCRIPTION OF THE INVENTION

Hereinafter, embodiments of the present invention will be described in detail so that those skilled in the art can easily implement the invention. However, the present invention may be implemented in various different forms and is not limited to the embodiments described herein.

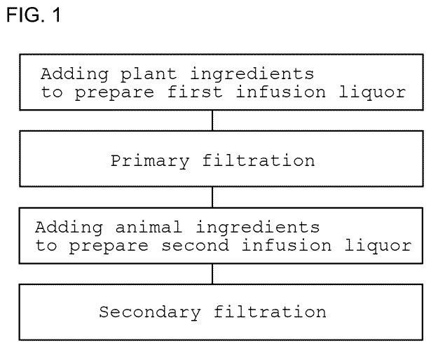

FIG. 1 is a schematic diagram illustrating a method of preparing a therapeutic agent for osteoporosis and plantar fasciitis according to one embodiment of the present invention.

Hereinafter, the therapeutic agent for osteoporosis and plantar fasciitis and method of preparing the same according to one embodiment of the present invention will be described in detail with reference to the attached FIG. 1.

The various plant and animal ingredients described below may be contained in themselves or may be present as ingredients extracted from the plant or animal ingredients into distilled liquor after soaked in distilled liquor for a predetermined period of time.

According to one embodiment of the present invention, a therapeutic agent for osteoporosis and plantar fasciitis contains pine roots, Fomitopsis pinicola, Fomitella fraxinea, Trametes versicolor, Sorbus commixta fruits, Phellinus linteus, Lespedeza bicolor petals, Lespedeza bicolor leaves, and distilled liquor.

The distilled liquor used herein may be, for example, clear soju. The present inventors used Chamisul Classic from Hite Jinro Co., Ltd., which has an alcohol content of 20.1%.

The pine root is also called “Songgeunbong” and has a round shape like a ball. In the present invention, fresh pine roots (Songgeunbong) were peeled and cut into a size of about 1 cm×1 cm×1 cm in width×length×height.

Meanwhile, Fomitopsis pinicola is also called “Cheonnyeonyeongji” and is a perennial mushroom belonging to the genus Polyporale and Fomitopsidaceae. It mainly grows on old coniferous trees or fallen trees, and causes brown rot. Administration of Fomitopsis pinicola extract has an effect of reducing blood sugar. In the present invention, fresh Fomitopsis pinicola was washed with clean water and cut into a size of about 1 cm×1 cm×1 cm in width×length×height for use.

Meanwhile, Fomitella fraxinea is also called “longevity mushroom” and is a mushroom belonging to the family Aphyllophorales. Lectin derived from Fomitella fraxinea is known to have excellent anticancer activity and immune activity. In the present invention, fresh Fomitella fraxinea was washed with clean water and cut into a size of about 1 cm×1 cm×1 cm in width×length×height and used.

Meanwhile, Trametes versicolor is also called “cloud mushroom” and is a mushroom belonging to the family Aphyllophorales, and is known to have preventive and therapeutic effects on hepatitis B, natural hepatitis, chronic active hepatitis, chronic bronchitis, and liver cancer, and is known to have therapeutic effects particularly for cancers of the digestive system, breast cancer, and lung cancer. In the present invention, fresh Trametes versicolor was washed with clear water and cut into a size of approximately 1 cm×1 cm×1 cm (length×width×height) for use.

Meanwhile, Sorbus commixta fruits are harvested from Sorbus commixta (Japanese rowan) which is a large deciduous tree of the rose family, grows in Korea, Japan, and the like, and is about 6-8 m tall. The bark is dark brown, the leaves are alternate and pinnately compound, and are composed of 4-7 pairs of leaflets, and the leaflets are long and oval with finely serrated edges. In early summer, small white flowers bloom in clusters in the leaf axils in a compound corymb, and the fruits are red and round. In the present invention, fresh Sorbus commixta fruits were washed with clear water and used without being cut.

Meanwhile, Phellinus linteus is also called “Meshimakobu” and is a very rare perennial mushroom of the basidiomycete family that grows (does not reproduce well) on old trees such as mulberry, oak, chestnut, and zelkova trees in mountainous areas. It mainly parasitizes on dead trees that have grown for several decades and is recorded in ancient books on oriental medicine such as “Bencao Gangmu” and “Donguibogam” under the names “Sang-I”, “Sangmoki”, “Sangsin” and “Chimyeolje”. The shape of Phellinus linteus resembles a lump of yellow mud in the beginning stage and then resembles a tongue sticking out of a tree stump when it fully grows. Therefore, it is also called “tree tongue” The upper part, which resembles a tongue, may be the color of mud or appear as black, cracked parts like the epidermis of a persimmon tree, the lower part may be like a yellow carpet, and the upper part may be black or mud-colored. When this mushroom is boiled in water, the color is yellow or light yellow, clear, and is tasteless. Phellinus linteus is broadly classified into five types, namely, Phellinus linteus, Fuscoporia gilva, Phellinus igniarius, Phellinus linteus, and Marasmiaceae. In Korea, eight types of Phellinus linteus out of about 50 types of Phellinus linteus exist in the world are known to grow naturally. Of these, Phellinus linteus (P. linteus, woody Sanghwang, mulberry tree Sanghwang) contains the most protein polysaccharides that exhibit immune components.

In the present invention, a lump of Phellinus linteus (overall size: approximately 45 cm×30 cm×14 cm in width×length×height) growing on Sorbus commixta trees was collected, thoroughly washed in clear water, and then cut into a size of approximately 1 cm×1 cm×1 cm in width×length×height for use.

Meanwhile, the petals and leaves of Lespedeza bicolor used in the present invention were each collected from Lespedeza bicolor. Lespedeza bicolor is a deciduous broadleaf shrub that is approximately 1-2 m tall, has a curved stem rather than a straight stem, and the leaves are opposite and trifoliate, and have petioles. The raceme on the leaves is longer than the leaves, unlike the true sedge. The small leaves are round and egg-shaped, have smooth edges, and are approximately 2-4 cm long. The flowers bloom in late summer and small reddish purple butterfly flowers bloom in a raceme. The fruits are flat and round legumes. One seed is formed inside the fruit. It is mainly found in mountainous areas and widely distributed throughout Korea. It is distributed outside Korea in Japan, northeastern China, and the Ussuri region. The flower blooms from July to August. In the present invention, the petals and leaves of Lespedeza bicolor collected from Lespedeza bicolor in August were used and the Lespedeza bicolor petals and leaves were each thoroughly washed with clean water and used without a separate cutting process.

The therapeutic agents for osteoporosis and plantar fasciitis according to the present invention is prepared by adding plant ingredients to distilled liquor and aging for a predetermined period of time, and the therapeutic agent for osteoporosis and plantar fasciitis thus prepared has an excellent effect of alleviating the symptoms of the disease in patients suffering from chronic osteoporosis and plantar fasciitis.

Meanwhile, the therapeutic agent for osteoporosis and plantar fasciitis according to the present invention may further contain various animal ingredients described below in addition to the plant ingredients.

That is, the therapeutic agent for osteoporosis and plantar fasciitis according to one embodiment of the present invention may further contain Cryptotympana atrata, Oxya sinuosa, Apis cerana Fabricius, Nephila clavata, Gryllus bimaculatus, Camponotus japonicus, Lumbricus terrestris, Pieris rapae, Sieboldius albardae Selys, and Acusta despecta sieboldiana.

As described above, the therapeutic agent for osteoporosis and plantar fasciitis according to the present invention including various plant ingredients and/or animal ingredients may be prepared by the method described below.

That is, the method of preparing the therapeutic agent for osteoporosis and plantar fasciitis according to the present invention includes preparing mixing pine roots, Fomitopsis pinicola, Fomitella fraxinea, Trametes versicolor, Sorbus commixta fruits, Phellinus linteus, Lespedeza bicolor petals, and Lespedeza bicolor leaves with distilled liquor to prepare a first mixture (S100), filtering the first mixture to remove solid ingredients (S200), adding Cryptotympana atrata, Oxya sinuosa, Apis cerana Fabricius, Nephila clavata, Gryllus bimaculatus, Camponotus japonicus, Lumbricus terrestris, Pieris rapae, Sieboldius albardae Selys and Acusta despecta sieboldiana to the filtered first mixture to prepare a second mixture (S300), and filtering the second mixture to remove the solid ingredients (S400).

More specifically, the method of preparing the therapeutic agent for osteoporosis and plantar fasciitis according to the present invention may include washing pine roots, Fomitopsis pinicola, Fomitella fraxinea, Trametes versicolor, and Phellinus linteus, cutting the same into a size of 1 cm×1 cm×1 cm in width×length×height, washing Sorbus commixta fruits, Lespedeza bicolor petals, and Lespedeza bicolor leaves, injecting the washed ingredients into a first glass container containing soju having an alcohol content of 20.1%, sealing the inlet of the first glass container, and storing the ingredients in an indoor storage space maintained at a temperature of 25° C. for 100 days, immediately after 100 days elapsed, opening the inlet of the first glass container and filtering the result through a 200-mesh strainer to remove the solid ingredients and thereby to obtain a first infusion liquor, injecting the filtered first infusion liquor in a second glass container that had been cleaned, adding 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana to the second glass container containing the first infusion liquor, sealing the inlet of the container, and storing the container in an indoor storage space maintained at a temperature of 25° C. for 100 days, and immediately after 100 days elapsed, opening the inlet of the second glass container and filtering the result through a 200-mesh strainer, to remove the solid ingredients and thereby to obtain a second infusion liquor.

The primary aging may be performed, for example, by injecting 6,000 g of pine roots, 100 g of Fomitopsis pinicola, 300 g of Fomitella fraxinea, 100 g of Trametes versicolor, Sorbus commixta fruits, Phellinus linteus, Lespedeza bicolor petals, and Lespedeza bicolor leaves prepared, as described above, into 50 L of Chamisul Classic from HiteJinro Co., Ltd. having an alcohol content of 20.1%, sealing the inlet of the container, and storing the container in an indoor storage space maintained at a temperature of 25° C. for 100 days.

Meanwhile, after the primary aging is completed, primary filtration of filtering the infusion liquor containing the plant ingredients may be performed through a strainer (200 mesh) to remove all the solid ingredients.

Then, the animal ingredients described below are added to the clear infusion liquor from which the solid ingredients have been removed by the primary filtration to perform secondary aging.

That is, the present invention is characterized in that the pine root, Fomitopsis pinicola, Fomitella fraxinea, Trametes versicolor, Sorbus commixta fruits, Phellinus linteus, Lespedeza bicolor petals, and Lespedeza bicolor leaves are added to distilled liquor such as soju, followed by aging the mixture for a predetermined period of time, to prepare a first infusion liquor using the plant ingredients, the first infusion liquor thus prepared is filtered to remove the solid ingredients, and then the animal ingredients described below are added to the clear first infusion liquor from which the solid ingredients have been removed and subjected to secondary aging. The present inventors found that, when the filtered and aged product obtained by separately performing primary aging using only plant ingredients and secondary aging using only animal ingredients, is consumed continuously for a long period of time, it is possible to obtain effects of improving bone density in osteoporosis patients, of ameliorating plantar muscle pain in plantar fasciitis patients, of improving cognitive function in patients with cognitive decline such as dementia, and of alleviating various menopausal symptoms in women as described above.

Hereinafter, secondary aging using animal ingredients will be described.

The secondary aging may be performed by adding Cryptotympana atrata, Oxya sinuosa, Apis cerana Fabricius, Nephila clavata, Gryllus bimaculatus, Camponotus japonicus, Lumbricus terrestris, Pieris rapae, Sieboldius albardae Selys, and Acusta despecta sieboldiana to the clear liquor from which the solid ingredients have been removed through the primary filtration, followed by aging for a predetermined period of time.

In order to prepare the animal ingredients, the present inventors bred the larvae and eggs of the insects, or the like, through a hygienic process of supplying limited food in an indoor breeding facility, and in particular, used the subjects obtained by breeding up to the third generation, and the third-generation subjects were obtained by selecting excellent subjects having no defect in appearance from subjects of each generation and repeatedly crossbreeding of the next generation.

The secondary aging according to the present invention may be performed by adding Cryptotympana atrata, Oxya sinuosa, Apis cerana Fabricius, Nephila clavata, Gryllus bimaculatus, Camponotus japonicus, Lumbricus terrestris, Pieris rapae, Sieboldius albardae Selys, and Acusta despecta sieboldiana to the clear infusion liquor from which the solid ingredients have been removed through the primary aging and primary filtration, followed by sealing the inlet of the container and storing the container in an indoor storage space maintained at a temperature of 25° C. for 100 days.

Meanwhile, the secondary aging may be performed by adding 7,000 to 9,000 g of Cryptotympana atrata, 3,000 to 5,000 g of Oxya sinuosa, 500 to 2,000 g of Apis cerana Fabricius, 2,000 to 3,000 g of Nephila clavata, 3,000 to 5,000 g of Gryllus bimaculatus, 500 to 700 g of Camponotus japonicus, 500 to 700 g of Lumbricus terrestris, 50 to 200 g of Pieris rapae, 50 to 200 g of Sieboldius albardae Selys, and 500 to 2,000 g of Acusta despecta sieboldiana, based on the first infusion liquor obtained through aging and filtration using 50 L of soju.

More specifically, the secondary aging is, for example, performed by weighing 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, washing the surfaces of the ingredients with clean water, and adding the result to the first infusion liquor obtained through aging and filtration using 50 L of soju, followed by sealing and storing for 100 days.

Meanwhile, the second infusion liquor obtained after the second aging period of 100 days has lapsed is poured into a strainer (200 mesh) to remove all solid ingredients, thereby performing secondary filtration.

Meanwhile, the infusion liquor using pine roots according to one embodiment of the present invention may be prepared according to the method of preparing the infusion liquor using pine roots according to various embodiments of the present invention.

Hereinafter, experiments demonstrating the effects of the present invention through examples of the present invention and various comparative examples will be described.

### Example 1

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which was surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

### [Example 2] Addition of Yeast Fermentation

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, 100 g of the yeast was added to the first infusion liquor, and fermentation was performed while the container lid was opened once a day for 10 minutes in an indoor storage space maintained at 25° C., and the container was sealed for the remaining time over 30 days. Then, the fermented first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 1] the same as in Example 1, except that pine roots were not added.

100 g of Fomitopsis pinicola, 300 g of Fomitella fraxinea, 100 g of Trametes versicolor, and 500 g of Phellinus linteus were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 2] the same as in Example 1, except that Fomitopsis pinicola was not added.

100 g of pine roots, 300 g of Fomitella fraxinea, 100 g of Trametes versicolor, and 500 g of Phellinus linteus were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 3] the same as in Example 1, except that Fomitella fraxinea was not added.

6,000 g of pine roots, 100 g of Fomitopsis pinicola, 100 g of Trametes versicolor, and 500 g of Phellinus linteus each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 4] the same as in Example 1, except that Trametes versicolor was not added.

6,000 g of pine roots, 100 g of Fomitopsis pinicola, 300 g of Fomitella fraxinea, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 5] the same as in Example 1, except that Phellinus linteus was not added.

6,000 g of pine roots, 100 g of Fomitopsis pinicola, 300 g of Fomitella fraxinea, and 100 g of Trametes versicolor, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which was surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 6] the same as in Example 1, except that Sorbus commixta fruits were not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 300 g of Lespedeza bicolor petals and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 7] the same as in Example 1, except that Lespedeza bicolor petals were not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 8] the same as in Example 1, except that Lespedeza bicolor leaves were not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits and 300 g of Lespedeza bicolor petals, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 9] the same as in Example 1, except that Cryptotympana atrata was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 10] the same as in Example 1, except that Oxya sinuosa was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 11] the same as in Example 1, except that Apis cerana Fabricius was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 12] the same as in Example 1, except that Nephila clavata was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 13] the same as in Example 1, except that Gryllus bimaculatus was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 14] the same as in Example 1, except that Camponotus japonicus was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 15] the same as in Example 1, except that Lumbricus terrestris was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 100 g of Pieris rapae, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 16] the same as in Example 1, except that Pieris rapae was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Sieboldius albardae Selys, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 17] the same as in Example 1, except that Sieboldius albardae Selys was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, and 1,000 g of Acusta despecta sieboldiana, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 18] the same as in Example 1, except that Acusta despecta sieboldiana was not added.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a second glass container that had been cleaned, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, and 100 g of Sieboldius albardae Selys, each of which had been surface-washed, were weighed and injected into the second glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

[Comparative Example 19]—simultaneous addition of plant ingredients and animal ingredients.

6,000 g of pine roots, 100 g of Fomitopsis pinicola, 300 g of Fomitella fraxinea, 100 g of Trametes versicolor, and 500 g of Phellinus linteus, each of which was freshly harvested, were washed, and cut into a size of 1 cm×1 cm×1 cm in width×length×height, 200 g of Sorbus commixta fruits, 300 g of Lespedeza bicolor petals, and 300 g of Lespedeza bicolor leaves, each of which was freshly harvested, were prepared, 8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed, all these ingredients were injected into a transparent glass container containing 50 L of Chamisul Classic from Hite Jinro Co., Ltd. having an alcohol content of 20.1%, the inlet of the container was sealed, and the container was stored in an indoor storage space maintained at a temperature of 25° C. for 200 days. Immediately after the 200 days have elapsed, the inlet of the container was opened, and the result was filtered through a 200-mesh strainer to remove the solid ingredients and thereby to obtain a first infusion liquor.

[Comparative Example 20]—Change of addition order of plant ingredients and animal ingredients

8,000 g of Cryptotympana atrata, 4,000 g of Oxya sinuosa, 1,000 g of Apis cerana Fabricius, 2,600 g of Nephila clavata, 4,000 g of Gryllus bimaculatus, 600 g of Camponotus japonicus, 600 g of Lumbricus terrestris, 100 g of Pieris rapae, 100 g of Sieboldius albardae Selys, and 1,000 g of Acusta despecta sieboldiana, each of which had been surface-washed, were weighed and injected into a first glass container containing 50 L of Chamisul Classic from Hite Jinro Co., Ltd. having an alcohol content of 20.1%, the inlet of the first glass container was sealed, and the first glass container was stored in an indoor storage space maintained at a temperature of 25° C. for 100 days. Immediately after 100 days elapsed, the inlet of the first glass container was opened, and the result was filtered through a 200-mesh strainer to remove the solid ingredients and thereby to obtain a first infusion liquor. Meanwhile, the filtered first infusion liquor was injected into a glass container that had been cleaned, 6,000 g of pine roots, 100 g of Fomitopsis pinicola, 300 g of Fomitella fraxinea, 100 g of Trametes versicolor, and 500 g of Phellinus linteus, each of which was freshly harvested, were washed and cut into a size of 1 cm×1 cm×1 cm in width×length×height, and 200 g of Sorbus commixta fruits, 300 g of Lespedeza bicolor petals, and 300 g of Lespedeza bicolor leaves, each of which was freshly harvested, were prepared. All the ingredients were injected into the glass container containing the first infusion liquor, the inlet of the container was sealed, and the container was stored in an indoor storage space maintained at a temperature of 25° C. for 100 days. Immediately after 100 days elapsed, the inlet of the second glass container was sealed and the result was filtered through a 200-mesh strainer, to remove the solid ingredients and thereby to obtain a second infusion liquor.

### [Experiment 1]—Test for Bone Density Improvement Effect

The following experiment was conducted on a total of 48 female test participants (46 osteoporosis patients and 2 normal subjects without osteoporosis) aged between 65 and 67 years. The purpose and content of the experiment were fully explained to test participants, consent was obtained for the experiment, and the experiment was conducted in accordance with medical practice and ethical standards. Bone density measurements were performed on the 48 test participants and the T-score, which is the bone density measurement result, is shown in Table 1 below (the average of the measurements of 2 participants for each experiment is shown). In Table 1 below, the normal group is the measured bone density of 2 normal subjects without osteoporosis and the control group is the measured bone density of subjects administering only soju without the therapeutic substances of examples and comparative examples. In the item “Day 0”, the bone density of the test participants without any test substance administration measured on the first day is shown and, in the item “1 week later”, the bone density of the test participants without any test substance administration measured after 7 days have passed since “Day 0”. This test was conducted for a total of six weeks.

Based on “0”, the normal value, as T-score increases toward+(plus), the bone density increases, and as T-score increases toward−(plus), the bone density decreases.

TABLE 1

| | | After 1 | After 2 | After 3 | After 4 | After 5 | After 6 |
|---|---|---|---|---|---|---|---|
| Item | Day 0 | week | weeks | weeks | weeks | weeks | weeks |
| Normal group* | +1.1 | +1.1 | +1.1 | +1.1 | +1.1 | +1.1 | +1.1 |
| Control | −2.1 | −2.1 | −2.1 | −2.1 | −2.1 | −2.1 | −2.1 |
| group** | | | | | | | |
| Example 1 | −2.1 | −2.0 | −1.5 | −1.1 | −0.5 | 0 | +1.2 |
| Example 2 | −2.1 | −2.0 | −1.4 | −0.8 | 0 | 1.0 | +1.5 |
| Comparative | −2.1 | −2.0 | −1.8 | −1.6 | −1.4 | −1.2 | −1.0 |
| Example 1 | | | | | | | |
| Comparative | −2.1 | −2.0 | −1.8 | −1.6 | −1.4 | −1.2 | −1.0 |
| Example 2 | | | | | | | |
| Comparative | −2.1 | −2.0 | −1.8 | −1.6 | −1.4 | −1.2 | −1.0 |
| Example 3 | | | | | | | |
| Comparative | −2.1 | −2.0 | −1.8 | −1.6 | −1.4 | −1.2 | −1.0 |
| Example 4 | | | | | | | |
| Comparative | −2.1 | −2.0 | −1.8 | −1.6 | −1.4 | −1.2 | −1.0 |
| Example 5 | | | | | | | |
| Comparative | −2.1 | −2.0 | −1.8 | −1.6 | −1.4 | −1.2 | −1.0 |
| Example 6 | | | | | | | |
| Comparative | −2.1 | −2.0 | −1.8 | −1.6 | −1.4 | −1.2 | −1.0 |
| Example 7 | | | | | | | |
| Comparative | −2.1 | −2.0 | −1.8 | −1.6 | −1.4 | −1.2 | −1.0 |
| Example 8 | | | | | | | |
| Comparative | −2.1 | −2.0 | −1.8 | −1.6 | −1.4 | −1.2 | −1.0 |
| Example 9 | | | | | | | |
| Comparative | −2.1 | −2.0 | −1.8 | −1.6 | −1.4 | −1.2 | −1.0 |
| Example 10 | | | | | | | |
| Comparative | −2.1 | −2.0 | −1.8 | −1.6 | −1.4 | −1.2 | −1.0 |
| Example 11 | | | | | | | |
| Comparative | −2.1 | −2.0 | −1.8 | −1.6 | −1.4 | −1.2 | −1.0 |
| Example 12 | | | | | | | |
| Comparative | −2.1 | −2.0 | −1.8 | −1.6 | −1.4 | −1.2 | −1.0 |
| Example 13 | | | | | | | |
| Comparative | −2.1 | −2.0 | −1.8 | −1.6 | −1.4 | −1.2 | −1.0 |
| Example 14 | | | | | | | |
| Comparative | −2.1 | −2.0 | −1.8 | −1.6 | −1.4 | −1.2 | −1.0 |
| Example 15 | | | | | | | |
| Comparative | −2.1 | −2.0 | −1.8 | −1.6 | −1.4 | −1.2 | −1.0 |
| Example 16 | | | | | | | |
| Comparative | −2.1 | −2.0 | −1.8 | −1.6 | −1.4 | −1.2 | −1.0 |
| Example 17 | | | | | | | |
| Comparative | −2.1 | −2.0 | −1.8 | −1.6 | −1.4 | −1.2 | −1.0 |
| Example 18 | | | | | | | |
| Comparative | −2.1 | −2.0 | −1.6 | −1.4 | −1.2 | −1.2 | −0.8 |
| Example 19 | | | | | | | |
| Comparative | −2.1 | −2.0 | −1.6 | −1.4 | −1.2 | −1.2 | −0.8 |
| Example 20 | | | | | | | |

### (Measured T-Score)

As can be seen from the results of Table 1 above, the bone density improvement effect of Examples 1 and 2 is superior to that of Comparative Examples 1 to 20, and in particular, the bone density improvement effect of Example 2 is superior to that of Example 1.

### [Experiment 2]—Test for Plantar Fasciitis Pain Amelioration Effect

The following experiment was conducted on a total of 48 female test participants (46 plantar fasciitis patients and 2 normal subjects without plantar fasciitis) aged between 55 and 57 years. The purpose and content of the experiment were fully explained to test participants, consent was obtained for the experiment, and the experiment was conducted in accordance with medical practice and ethical standards. The experiment was conducted by evaluating the level of pain in the plantar fascia area for 48 test participants on a scale of 1 to 10 and the results are shown in FIGS. 1 and 2 (average of the measured values of 2 participants for each experiment is shown).

In Table 2 below, the normal group is the pain measured on 2 normal subjects without plantar fasciitis and the control group is the pain measured on subjects administering only soju without the therapeutic substances of examples and comparative examples. In the item “Day 0”, the pain of the test participants without any test substance administration measured on the first day is shown and, in the item “after 1 week”, the pain of the test participants without any test substance administration measured after 7 days have passed since “Day 0”. This test was conducted for a total of six weeks. The pain 1 means there is no pain at all in the related area. As the number increases from 2 to 10, the level of pain increases.

The experiment was conducted using the following numerical pain rating scale which corresponds to the criteria used as an indicator for selecting appropriate painkillers in the World Health Organization (WHO) 3-step analgesic use guidelines.

That is, this numeric rating scale indicates the level of pain on a basis of scale from 0 to 10. “0” means no pain and “10” means unimaginably severe pain. The test participants were asked to mark the number from 0 to 10 that best represented how severe their current pain was, and on this test scale, pain from 1 to 10 is classified as mild (1 to 3), moderate (4 to 6), and severe (7 to 10).

TABLE 2

| | | After 1 | After 2 | After 3 | After 4 | After 5 | After 6 |
|---|---|---|---|---|---|---|---|
| Item | Day 0 | week | weeks | weeks | weeks | weeks | weeks |
| Normal group* | 0 | 0 | 0 | 0 | 0 | 0 | 0 |
| Control | 4.5 | 4.5 | 4.5 | 4.5 | 4.5 | 4.5 | 4.5 |
| group** | | | | | | | |
| Example 1 | 5 | 4.5 | 4 | 3.5 | 3 | 2 | 1.5 |
| Example 2 | 5 | 3.5 | 3.0 | 2.5 | 2 | 1.5 | 0.5 |
| Comparative | 5 | 4.5 | 4.5 | 4 | 4 | 3.5 | 3.5 |
| Example 1 | | | | | | | |
| Comparative | 4 | 3.5 | 3.5 | 3 | 3 | 3 | 2.5 |
| Example 2 | | | | | | | |
| Comparative | 5 | 4.5 | 4.5 | 4 | 4 | 4 | 3.5 |
| Example 3 | | | | | | | |
| Comparative | 5 | 4.5 | 4.5 | 4 | 4 | 4 | 3.5 |
| Example 4 | | | | | | | |
| Comparative | 4.5 | 4 | 4 | 3.5 | 3.5 | 3.5 | 3 |
| Example 5 | | | | | | | |
| Comparative | 5 | 4.5 | 4 | 3.5 | 3.5 | 3.5 | 3 |
| Example 6 | | | | | | | |
| Comparative | 5 | 4.5 | 4 | 3.5 | 3.5 | 3.5 | 3 |
| Example 7 | | | | | | | |
| Comparative | 5 | 4.5 | 4 | 3.5 | 3.5 | 3.5 | 3 |
| Example 8 | | | | | | | |
| Comparative | 5 | 4.5 | 4 | 3.5 | 3.5 | 3.5 | 3 |
| Example 9 | | | | | | | |
| Comparative | 4 | 3.5 | 3.5 | 3 | 3 | 3 | 2.5 |
| Example 10 | | | | | | | |
| Comparative | 5 | 4.5 | 4 | 3.5 | 3.5 | 3.5 | 3 |
| Example 11 | | | | | | | |
| Comparative | 5 | 4.5 | 4 | 3.5 | 3.5 | 3.5 | 3 |
| Example 12 | | | | | | | |
| Comparative | 4.5 | 4 | 4 | 3.5 | 3.5 | 3 | 3 |
| Example 13 | | | | | | | |
| Comparative | 5 | 4.5 | 4 | 3.5 | 3.5 | 3.5 | 3 |
| Example 14 | | | | | | | |
| Comparative | 4 | 4 | 3.5 | 3.5 | 3.5 | 3.5 | 3 |
| Example 15 | | | | | | | |
| Comparative | 5 | 4.5 | 4 | 3.5 | 3.5 | 3.5 | 3 |
| Example 16 | | | | | | | |
| Comparative | 4 | 4 | 4 | 3.5 | 3.5 | 3.5 | 3 |
| Example 17 | | | | | | | |
| Comparative | 5 | 4.5 | 4 | 3.5 | 3.5 | 3.5 | 3 |
| Example 18 | | | | | | | |
| Comparative | 5 | 4 | 3.5 | 3 | 3 | 3 | 2.5 |
| Example 19 | | | | | | | |
| Comparative | 5.5 | 4.5 | 3.5 | 3 | 3 | 3 | 2.5 |
| Example 20 | | | | | | | |

As can be seen from the results of Table 2 above, the plantar fasciitis pain amelioration effect of Examples 1 and 2 is superior to that of Comparative Examples 1 to 20, and in particular, the plantar fasciitis pain amelioration effect of Example 2 is superior to that of Example 1.

### [Safety Test]

During the process of conducting Experiments 1 and 2, the experimental participants were surveyed every week for 6 weeks during the experiment and for an additional 6 weeks after the experiment ended to determine whether or not they had any physical or mental abnormalities in each body part. No physical or mental abnormalities were observed at all in all the test participants related to Experiments 1 and 2 and these results verify that the test composition according to the present invention is safe.

As is apparent from the above description, the therapeutic agent for osteoporosis and plantar fasciitis and the method of preparing the same according to the present invention have the effects of reducing the rate of decrease in bone density in patients with osteoporosis or increasing the bone density in patients with osteoporosis, significantly ameliorating heel pain in patients with plantar fasciitis, improving cognitive ability in elderly people with dementia, and alleviating various menopausal symptoms in women.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A therapeutic agent for osteoporosis and plantar fasciitis comprising:
pine roots;
Fomitopsis pinicola;
Fomitella fraxinea;
Trametes versicolor;
Sorbus commixta fruits;
Phellinus linteus;
Lespedeza bicolor petals;
Lespedeza bicolor leaves; and
distilled liquor.

2. The therapeutic agent according to claim 1, further comprising:
Cryptotympana atrata;
Oxya sinuosa;
Apis cerana Fabricius;
Nephila clavate;
Gryllus bimaculatus;
Camponotus japonicus;
Lumbricus terrestris;
Pieris rapae;
Sieboldius albardae Selys; and
Acusta despecta sieboldiana.

3. A method of preparing a therapeutic agent for osteoporosis and plantar fasciitis comprising:
preparing mixing pine roots, Fomitopsis pinicola, Fomitella fraxinea, Trametes versicolor, Sorbus commixta fruits, Phellinus linteus, Lespedeza bicolor petals, and Lespedeza bicolor leaves with distilled liquor to prepare a first mixture (S100);
filtering the first mixture to remove solid ingredients (S200);
adding Cryptotympana atrata, Oxya sinuosa, Apis cerana Fabricius, Nephila clavata, Gryllus bimaculatus, Camponotus japonicus, Lumbricus terrestris, Pieris rapae, Sieboldius albardae Selys and Acusta despecta sieboldiana to the filtered first mixture to prepare a second mixture (S300); and
filtering the second mixture to remove solid ingredients (S400).
